(19) United States
(12) Patent Application Publication
(10) Pub. No.: US 2013/0131328 A1
(43) Pub. Date: May 23, 2013

(54) PROCESS FOR PRODUCING REFINED NUTRACEUTIC EXTRACTS FROM ARTICHOKE WASTE AND FROM OTHER PLANTS OF THE CYNARA GENUS

(71) Applicants: ISR Ecoindustria S.R.L., Latina (IT); Massimo Pizzichini, Roma (IT); Annalisa Romani, S. Michele Agliana (IT)

(72) Inventors: Massimo Pizzichini, Roma (IT); Annalisa Romani, S. Michele Agliana PT (IT); Daniele Pizzichini, Roma (IT); Claudio Russo, Roma (IT); Patrizia Pinelli, Fiorentino (FI) (IT)

(73) Assignees: Massimo PIZZICHINI, Romo (IT); ISR ECOINDUSTRIA S.R.L., Latina (IT); Annalisa ROMANI, S. Michele Agliana (IT)

(21) Appl. No.: 13/669,257

(22) Filed: Nov. 5, 2012

Related U.S. Application Data
(63) Continuation of application No. 12/529,002, filed on Oct. 27, 2009, now abandoned.

(30) Foreign Application Priority Data
Feb. 28, 2007 (IT) RM2007A000109

(51) Int. Cl. C07H 1/08; C07C 69/618; C07H 15/26; C07D 311/30; C07C 67/48; C07D 311/40

(52) U.S. Cl. CPC C07H 1/08; C07C 67/48; C07D 311/40; C07H 15/26; C07D 311/30; C07C 69/618
USPC 536/18.1; 560/75; 549/403

(57) ABSTRACT

Process for fractioning and refining natural products obtainable from waste vegetal material and particularly from artichoke (Cynara scolymus) production or from other plants of the Cynara genus such as the cultivated or wild cardoon. The process is based on the use of membrane separation technologies envisaging a tangential microfiltration (MF) phase on the raw decoction, followed by tangential ultrafiltration (UF) on the previous MF permeate and reverse osmosis (RO) on the UF permeate, in order to obtain a retentate rich in concentrated active ingredients and a permeate consisting of ultrapure water that is recycled for the preparation of the decoction.

The process enables obtaining purified extracts of high biological valence to be used in the pharmaceutical industry, in the nutraceutic sector, in the cosmetics industry and for innovative products in the food industry.

The present invention concerns a process for producing refined nutraceutic extracts from artichoke waste and from other plants of the Cynara genus. More specifically, the invention concerns a process for fractioning and refining natural products obtainable from waste vegetal material, and particularly the waste of artichoke production, or from other vegetal material coming from the Cynara genus, such as the cardoon. The process is based on the use of membrane-based separation technologies and enables producing purified extracts of high biological valence to be used in the pharmaceutical industry, in the nutraceutical field, in the cosmetics industry or for innovative products in the food industry.

As is known, the Cynara genus, which belongs to the Compositae family, includes the species Cynara cardunculus which in turn includes the artichoke (C. cardunculus L. subsp. scolymus L., normally referred to as Cynara scolymus), the cultivated cardoon (C. cardunculus L. var. altilis DC), the wild cardoon (C. cardunculus L. var. sylvestris Lam.) and some other lesser known species. The inflorescence of these plants, the heads, are made up of many single flowers, of a purplish colour when blooming, which are gathered in the central part by a receptacle or thalamus, around the outermost part of which there are the bracts or involucral scales.

When they are immature and tender, the heads of the Cynara scolymus are harvested, before the bracts harden and before the central flowers grow too much. They provide the edible part of the artichoke, commonly known as the “heart”, consisting of the receptacle and the florets it contains, as well as of the fleshy bases of the internal bracts, and possibly of a part of the peduncle.

The artichoke is a characteristic Italian product, although in recent years its cultivation has also extended to other geographic areas (such as California, Argentina and New Zealand), and it has been estimated that the Mediterranean area has 90% of the world's production, with Italy being the leading producer with an estimated production well over 500,000 tons/year. Although Italy has considerable land given over to artichoke production, there are also notable productions in Spain and France.

Italian production is mainly destined for fresh consumption, even if the quantity destined for the food processing industry is growing, especially as regards the production of oil-preserved foodstuffs.

Unlike many other cultivations, in artichoke growing the relation between the edible part and the whole plant is extremely low (about 15%), and thus the quantity of by-product is considerable. The artichoke heart accounts for 40-55% of the weight of the whole head and contains about 15% of dry matter, and is particularly rich in fibre. The remaining part, consisting of leaves and stems, constitutes the waste material that must often be discarded with additional waste treatment costs in compliance with environmental laws. This waste matter could be widely and advantageously used both as a semi-processed component in human foods and as animal feed. The use of waste matter and residues deriving from the industrial agronomic sector of artichoke production and its derivatives could enable a reduction in the environmental impact of the production itself, and the reduction of labour costs and biomass disposal costs.

Although the current state of the art is essentially geared to their conversion into energy, rarely are these biomasses considered to represent new low-cost raw materials for the production of bio-products of high added value for the food, phytotherapy and cosmetics industry.

The total raw fibre of the artichoke, consisting of 65% cellulose, 21% semi-cellulose and 14% lignin, accounts for an overall 1.4% of the fresh substance. From a nutritional and dietary point of view, the artichoke is a noble food for the abundance of azotised substances and for its carbohydrate content. In essence, it is a vegetable of average energy value (38 cal/100 g), not only rich in mineral salts (K, Na, Ca, P, Fe), vitamins (A, B₁, C) and sugars (including inulin, a fructose polymer of pre-biotic activity, which promotes the proper equilibrium of bowel bacterial flora), but also—and especially—polyphenol compounds responsible for considerable biological activity for which the artichoke has been renowned as an officinal plant since ancient times.

In fact, artichoke extracts and dyes, which are mostly obtained from the plant's leaves, have long been used as hepatoprotectors and regulators of liver functioning, in hepatobiliary pathologies, as choleretics (to increase biliary flow) and hypocholesterolemics (to lower cholesterol levels in the blood). In general, both the spontaneous and cultivated forms of the Cynara genus are used in order to extract biopharmaceutical substances.

The different compounds of vegetal origin contained in them can express different biological properties (antioxidant, anti-radical, antimicrobial) and in many cases, the single molecule is less active with respect to the mixture of compounds, thus suggesting a synergic action among them. In other words, it is not rare to find that raw extracts of vegetal origin have a higher bioactivity with respect to the one found when individual compounds in these plants are used alone.

The polyphenol compounds responsible for the biological activities of the artichoke include: a) the hydroxycinnamates of the chlorogenic acid family, among which cholorogenic or mono-caffeoylquinic acid (5-O-caffeoylquinic), dicaffeoylquinic or cynarin acid (1,5-dicaffeoylquinic acid); b) flavonoids and flavonoic eterosides (luteolin and its conjugates, such as cynaroside (luteolin-7-O-glucoside), scolimoside, luteolin-glucuronide; c) lactone sesquiterpens, among which the compounds responsible for the characteristic bitter taste, cynaropicrin and dehydrocynaropicrin.

The Cynara thus has important applications not only in phytotherapy but also in the cosmetics industry, where the extracts are used for their antioxidant and anti-free radical properties.

In many pharmacological studies, the Cynara extracts have been found to possess various properties: (i) they protect proteins, lipids and DNA from oxidation caused by free radicals; (ii) they show a choleretic, diuretic and hepatoprotective activity; (iii) they inhibit the biosynthesis of cholesterol contributing to the prevention of arteriosclerosis and other circulatory disorders; (iv) they inhibit HIV integrase, the key enzyme in HIV replication and in its integration in the host genoma; and (v) they possess antibacterial activity.

Recent studies (Romani A., Pinelli P., Cantini C., Cimato A. & Heimler D. “Characterization of Violetto di Toscana, a typical Italian variety of artichoke (Cynara scolymus L.)”, Food Chemistry, 95, 221-225, 2006) on cultivars of Violetto di Toscana and Terom, the latter widely available on the market, have characterised the polyphenol content of various Cynara tissues (leaves, bracts, heads, stems) and it has been found that the stem has a polyphenol composition very similar to that of the head, that is, the edible part of the artichoke. These tissues are essentially rich in caffeoylquinic derivatives, while the leaves and bracts are also rich in flavonoid compounds.

The antioxidant properties in human LDL (low density lipoproteins) of extracts obtained from various artichoke tissues have also been evaluated (Coinu R., Carta S., Urgeghe P. P., Mulinacci N., Pinelli P., Franconi F., Romani A. “Dose-Effect study on the antioxidant properties of leaves and outer bracts of extracts obtained from Violetto di Toscana artichoke”, Food Chem., 2007, 101, 524-531) for which a high activity has always been recorded. The studies have also been extended to cultivated cardoon and its progenitor, the wild cardoon, both belonging to the Cynara genus. These species contain polyphenol antioxidants similar to those found in the artichoke (caffeoylquinic acids and flavonoids) (Pinelli P., Ieri F., Buzzini P., Turchetti B., Lanteri S., Romani A., “Composti polifenolici ad attività antimicotica in tessuti di diverse cultivar di carciofo”, VII CISETA (Congresso Italiano di Scienza E Tecnologia degli Alimenti), Cernobbio (CO), 19-20 Settembre 2005). The cardoon also contains hydroxycinnamic compounds which are either absent or found only in traces in the artichoke (Pinelli P., Agostini F., Comino C., Lanteri S., Portis E., Romani A. “Polyphenolic Composition of Wild and Cultivated Cardoon Leaves”, 2007, forthcoming publication).

In the more specifically phytotherapeutic field, it is known that artichoke extracts are obtained by extraction with water or with hydroalcoholic solutions from either fresh or dried leaves. For example, US patent application 2004/0234674 (Eich et al.) describes artichoke leaf extracts characterised by a total content of caffeoylquinic acids (mono- and dicaffeoylquinics of at least 6% with respect to the total quantity of the extract, and a total content of flavonoids of at least 3%, still with respect to the total quantity of extract. According to the description, these products were obtained by means of liquid-liquid extraction of a primary extract from fresh or dried leaves of Cynara, in which the extraction solvent is an organic non-aqueous solvent, and the resulting aqueous phase is the one that is recovered.

Still in the same field, the international patent application No WO 2007/006391 (Indena S.p.A.) describes Cynara scolymus extracts obtainable by fractionation on resins, in which the above-surface parts of the plants (including the heads), both in fresh and dried state, are subjected to extraction with a hydroalcoholic solution to which a quantity of cysteine is added in order to obtain a large quantity of cynaropicrin in the final extract, and to maintain this cynaropicrin stable in therapeutic formulations. Sulfurated amino acids are deemed to give rise to adducts that stabilise the sesquiterpen. The hydroalcoholic extracts are then concentrated, and the precipitated substances are separated by filtration, and the resulting solution is concentrated and purified on an adsorption resin. Finally, the desired extract is eluted with ethanol from the resin.

Turning back to the agro-food sector, it must be noted that not only consumption of the fresh vegetable, but also—and especially—its processing and packaging produces a high quantity of waste (leaves, stems and waste water). In the artichoke processing industry, in some cases this waste exceeds 60% of the overall vegetal massa Over and beyond its uses in phytotherapy and cosmetics, the extracts from artichoke and cardoon wastes can be used as additives in order to improve the quality of both animal feeds and human foodstuffs (lowering lipid peroxidation and increasing the health properties of the foods themselves, as well as providing fibre). Moreover, thanks to the antimicrobial properties of the artichoke, the leaf extracts are considered as potential additives in foods for which natural protection is sought (biocontrol) against polluting microbes.

In view of the above, an object of the present invention is to provide a production system enabling the advantageous reuse of the waste products of artichoke production and possibly other vegetal materials or wastes from specifically cultivated plants belonging to the Cynara genus, in order to obtain purified extracts, both in powdered and liquid form, useful for their active ingredients to be used in the agro-food industry as well as in nutraceutic, phytotherapeutic, cosmetic and dermatological products.

To this end, a production method has been devised, according to the present invention, for extracts of artichoke waste, particularly but not exclusively from the plant leaves, and based on a refining process using membrane-based separation technologies. As is known, these technologies are a safe, reliable and innovative alternative (Best Available Technology) to the traditional solvent-based extraction techniques. These techniques do not in troduce contaminating substances (and, in particular, do not use organic solvents), work at room temperature and thus do not thermally damage the matrix, are perfectly sterilizable, and are thus microbiologically safe, and have also been widely tried and tested in the most delicate pharmaceutical preparations.

The proposed process is based on the specialist use of membrane-based separation technologies, applied directly to a raw decoction, in water, of vegetal materials to be treated, after separating the solid material from it by means of mechanic filtration. The filtered liquid undergoes a tangential microfiltration (MF) phase, a tangential ultrafiltration (UF) phase on the previous permeate, whose permeated product yields practically the total amount of nutraceutic active ingredients of Cynara, and a reverse osmosis (RO) phase on the UF permeate, which yields a retentate rich in concentrated active ingredients and a permeate, consisting of ultrapure water, that is recycled to the previous extraction phases, and in the preparation of the decoction bath.

For some of the uses envisaged, the RO concentrate is dried or freeze-dried to obtain a stable powder rich in polyphenol antioxidant molecules, in particular, flavonoids (such as the glucosides of luteolin and apigenin) and caffeoylquinic esters (such as chlorogenic acid and cynarin).

Therefore, the present invention specifically provides a process for the production of refined extracts from artichoke waste and from other plants of the Cynara genus, comprising, in sequence, the following operations:
a) obtaining a decoction of vegetable material from plants of the Cynara genus, in water, by means of hot infusion (that is, at temperatures higher than room temperature, but lower than boiling point);
b) mechanically separating the vegetal material obtained in the previous phase from the filtered liquid phase;
c) treating the said filtered liquid phase resulting from operation b) by means of tangential microfiltration (MF), to yield a retentate phase and a permeate phase;
d) treating the permeate coming from the previous operation by means of tangential ultrafiltration (UF), to yield a retentate phase and a permeate phase;
e) treating the permeate coming from operation d) by means of reverse osmosis (RO), to yield a retentate phase rich in purified polyphenol active ingredients and a permeate phase consisting of demineralised water;
   the said vegetal material decoction separated from phase b), being depleted of bitter polyphenol compounds, and being reusable as animal feed, and the said MF and UF retentate phases coming from operations c) and d) being reusable in the human food sector.

Preferably, the reverse osmosis concentrate, rich in purified polyphenol active ingredients, is spray-dried or freeze-dried to obtain a stable powder rich in antioxidant molecules.

With specific reference to the case in which the vegetal material to be treated is composed of waste of the artichoke production process, and specifically the leaves, outer bracts and/or stems or even heads that are considered inedible, this material can be used after its separation into the various types of tissues or mixed and, as already noted with reference to the scientific literature published or in publication, will give rise to refined products of an overall different chemical composition. In essence, refined products obtained from stems or from heads have very similar active ingredient compositions, with a prevalence of caffeoylquinic compounds, while leaves and bracts are also rich in flavonoid derivatives.

As already noted, the process of the invention can be applied not only to vegetal material selected from artichoke leaves, outer bracts, stems and heads taken separately or intermixed, but also to the case where the vegetal material comes from cultivated cardoon, C. cardunculus L. var. altilis, or even from wild cardoon, C. cardunculus L. var. sylvestris. In all these cases, the active ingredients recoverable from the ultrafiltration operation belong to correlated polyphenol families, even if, depending on the botanical variety and also on the cultivar and cultivation and harvesting conditions, the tenors of the individual compounds belonging to the main families will be different.

Preferably, the hot-infusion of the vegetal material is carried out by heating the said material in water (tap or distilled water) at a temperature between 80° C. and 95° C., and preferably 85° C., with a weight/volume ratio between 10 and 35%, for an overall time ranging between 15 and 45 minutes, and preferably about 30 minutes. At the end of heating in an aqueous medium, the decoction is collected by mechanic filtration on a 2-10 mm mesh filter, and preferably a 2 mm stainless steel mesh filter.

The artichoke leaves and stems may undergo a second hot extraction phase to totally remove the residual active ingredients. In this case, after phase b) of mechanical separation, the separated vegetal material undergoes a second hot-infusion phase, followed by a further mechanical separation of the vegetal material obtained, to yield a filtrated liquid phase.

The residual vegetal part that is collected after decoction is depleted or completely devoid (second extraction) of polyphenol and caffeoylquinic compounds, which are particularly bitter, normally present in Cynara, and may thus be an interesting raw material for cattle feeds, also by adding other components (forage, purple medic, clover, etc.).

The filtered liquid of the decoction is then cooled to 30° C. and treated with three different membrane-based tangential filtration technologies: microfiltration, ultrafiltration and reverse osmosis.

Operation c) of tangential microfiltration can be carried out with spiral-wound polymer membranes or ceramic membranes, with a molecular size in the range 0.10-3.0 μm. The said spiral-wound polymer membranes are preferably made of polyethersulfone, regenerated cellulose acetate or nylon, while the ceramic ones are preferably made of a tubular ceramic monolith of alumina with an internal coating of zirconia or titanium oxide, of the isoflux type, of a “sunflower” or “dahlia” shape, with 23 or 39 filtration channels.

According to the preferred embodiments of the invention, downstream of the said operation c) of MF a diafiltration is carried out, feeding the membrane with demineralised water (RO permeate), that is added to the MF retentate.

Operation d) of tangential ultrafiltration is preferably carried out with polymer membranes of molecular cut-off between 500 Dalton and 50 kDalton. The polymer membranes for UF are of the spiral type and are made of one of the following materials: polysulfone, polyethersulfone, nylon or regenerated cellulose acetate.

Still according to a preferred solution of the present invention, downstream of operation d) of UF a diafiltration is carried out, feeding the membrane with purified water (RO permeate), obtained from the said operation e) of reverse osmosis, that is added to the UF retentate.

As already noted, the reverse osmosis operation is carried out in order to eliminate the water (permeate) and to concentrate the active ingredients of Cynara, and may be conducted with spiral-wound polymer membranes with low or high saline rejection normally at pressures ranging between 7 and 50 bar, and at feed flow rates between 0.5 and 2 m³/h, when 4×40 inch (10.16 cm×101.6 cm) spiral type modules are used.

According to some forms of specific realisations of the invention, the membranes used are polymer membranes of various chemical nature, geometrical shape (flat, hollow-fibre, spiral-wound, tubular, boxed, etc.) and size of the modules.

If desired, the polyphenol active ingredients of the RO concentrate can be directly exploited with the product in liquid form, or they can be dried in order to obtain a powder. Preferably, the said reverse osmosis retentate drying operation is carried out by spray-drying, possibly after adding dextrans or maltodextrins (preferably 30 g/L) to the RO retentate in order to improve the texture and stability of the powder.

The refined extracts based on the purified polyphenol active ingredients obtainable from the process proposed according to the present invention are advantageously used both for food production and as nutraceutic, pharmaceutical, phytotherapeutic and cosmetic products. In particular, these extracts can be used in even innovative type food products such as special dough for baking products and/or powdered products or, in liquid form, as stabilising additives for foodstuffs, such as for stabilising (in lieu of ascorbic acid) “fresh” vegetal products such as artichoke hearts that are used, unpreserved, in the catering industry.

In solid powdered form, the refined extracts of Cynara prepared according to the present invention are useful not only as nutraceutic and pharmaceutical active ingredients, such as for over-the-counter (OTC) products, but also as semi-finished components of antioxidant and anti-free radical activity for cosmetics and dermatological products.

In short, the production system according to the invention is characterised by the following advantageous aspects:
it uses an innovative process enabling the recovery and reuse of all the chemical components resulting from the treatment of the wastes of artichoke production;
it respects the environment, also by the integrated reuse of secondary effluents, including osmotised water;
it avoids the use of organic solvents in the extraction, which would negatively affect the use of the recovered active ingredients in foodstuffs and pharmaceuticals;
it enables the development of an industrial process of a scale consistent with the quantities of raw material available in the national territory owing to the membrane technologies, which are modular and thus easily adaptable to any scale of production;
it allows using a range of purified, concentrated active ingredients, formulated in stabilised form.

The specific characteristics of the present invention, as well as its advantages and relative operational modalities, will be more evident with reference to the detailed description presented merely for exemplificative purposes below, along with the results of the experimentations carried out on it. The diagrams of the proposed process and some experimental results are also illustrated in the attached drawings, wherein:

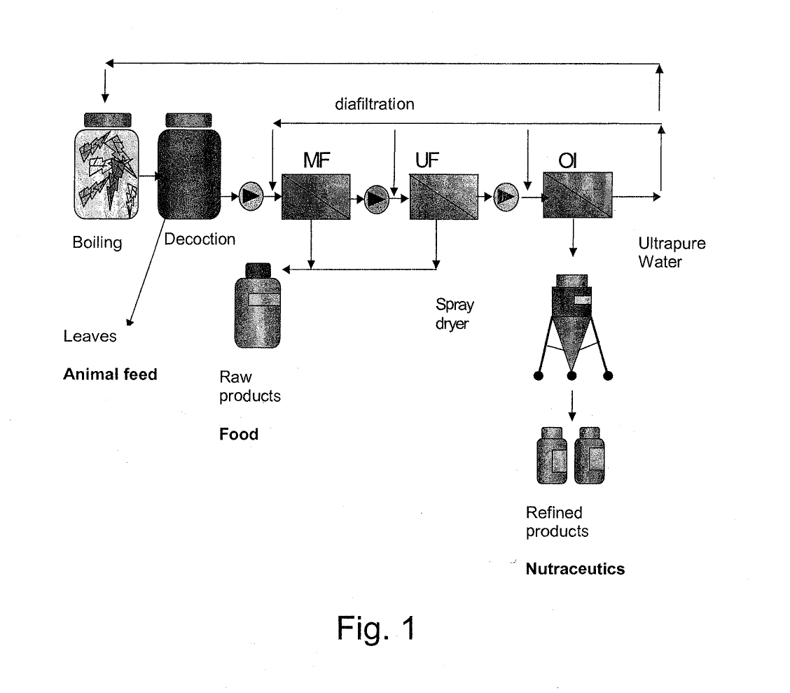

FIG. 1 shows a block diagram of the extraction and refining process from vegetal material of the Cynara genus proposed according to the present invention;

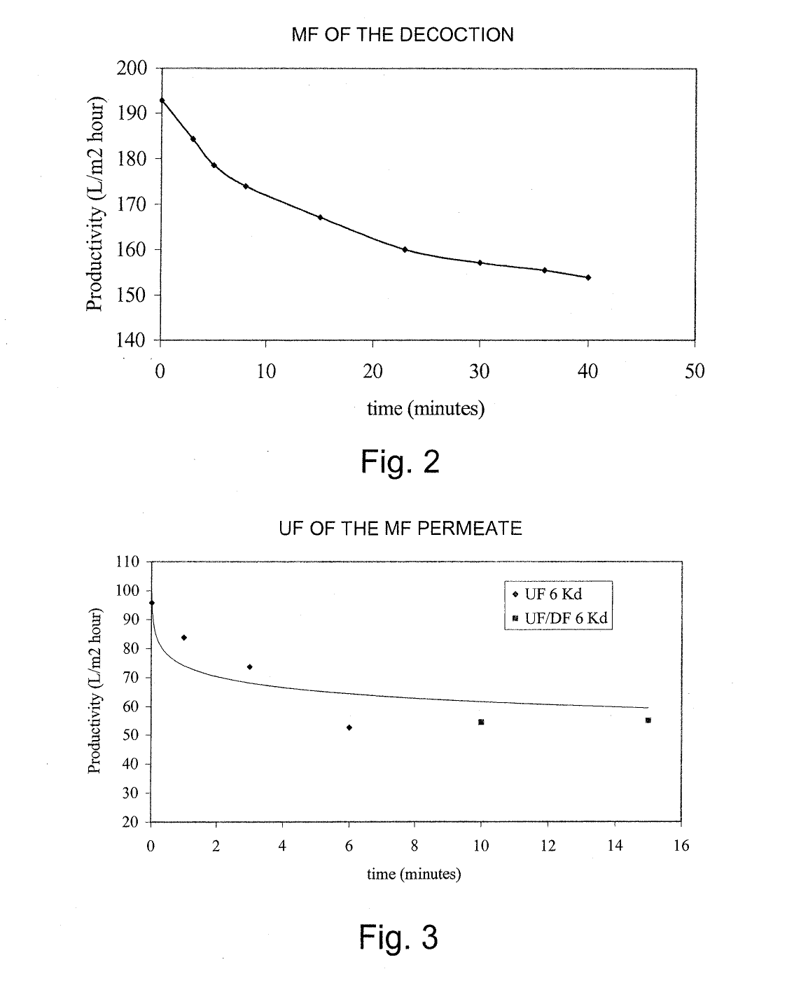

FIG. 2 shows the trend over time of the permeate flow through the microfiltration (MF) membrane of the Example;

FIG. 3 shows the trend over time of the permeate flow through the ultrafiltration (UF) membrane of the Example;

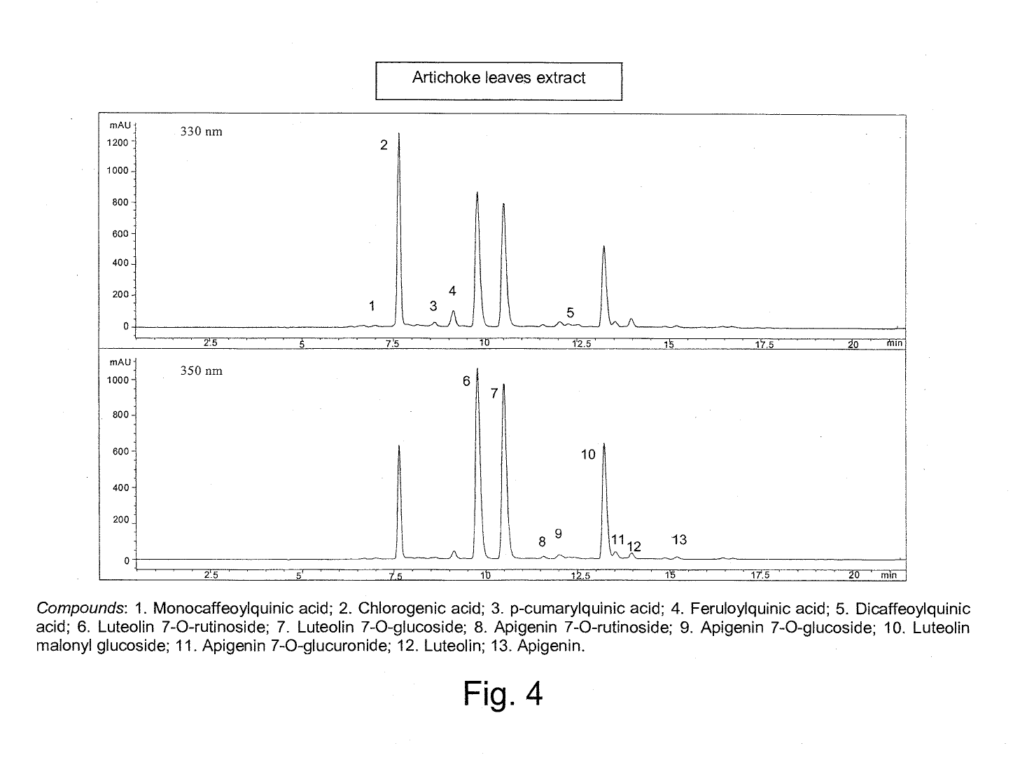

FIG. 4 shows the HPLC chromatogram of the artichoke extract powder obtained as the final product from the process of the Example, with the legend of the polyphenol compounds present; and

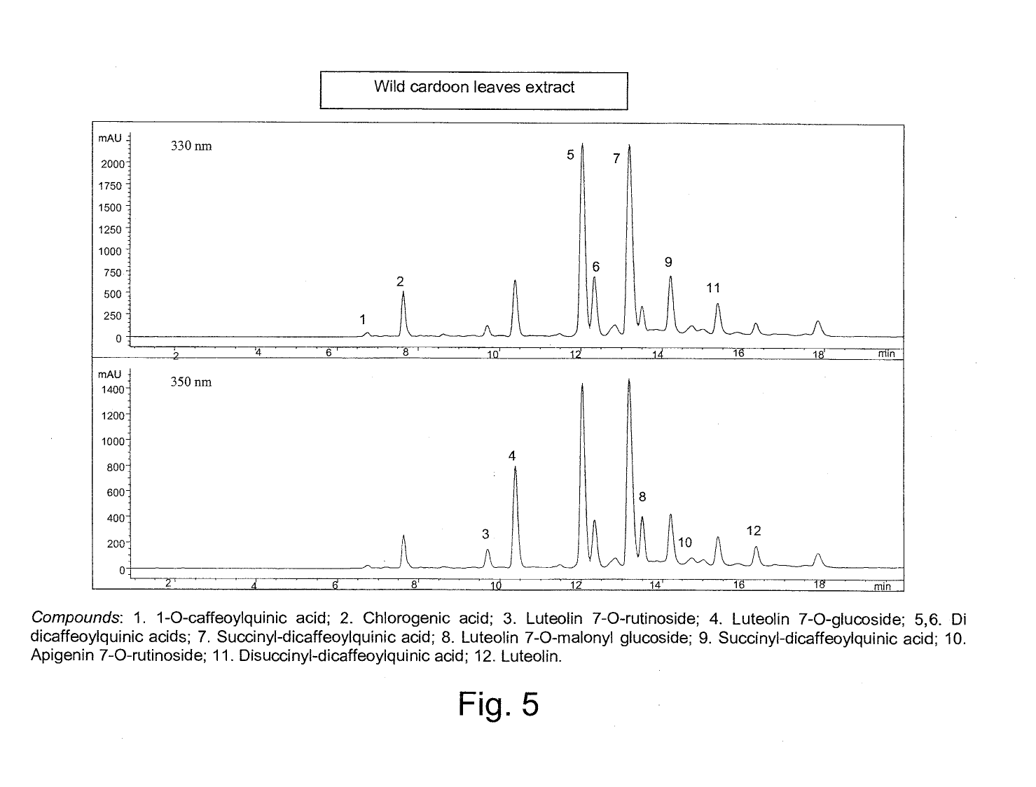

FIG. 5 shows the HPLC chromatogram of the powder obtained from a wild cardoon extract, with the legend of the polyphenol compounds present.

### EXAMPLE

The overall scheme of the process applied is the same as the one shown in FIG. 1.

### Decoction of the Vegetal Material

The aqueous extract of artichoke leaves is obtained by heating the vegetal material (leaves, bracts, stems, the heads considered inedible for their size, etc.) in water at 85° C. for about 30 minutes, avoiding boiling.

In this case, 25 kg of fresh vegetal material (leaves) was used in 100 L of tap water (25% dry weight), with no particular requirements of chemical purity. The decoction solution is separated from the leaves by simple filtering on 2 mm wire mesh and then fed to tangential microfiltration. This solution was characterised as regards the biophenol content of the starting extract (the input datum is necessary both for standardising the extraction conditions and for defining the characteristics of the finished product).

Microfiltration of the Decoction, with 0.14 μm Molecular Cut-Off

The aqueous extract was microfiltered (MF) with alumina tubular ceramic membranes, internally coated with zirconia, of the isoflux type by Tami (France), and of a “sunflower” shape (23 channels, 3.6 mm in diameter) with a porosity of 0.14 μm and filtering surface of 0.35 m².

The process is carried out with a pilot apparatus of the ENEA Casaccia laboratories that uses two ceramic membranes in parallel of the aforesaid type. The process parameters of the MF trial are reported in Table 1 below.

TABLE 1
Process parameters of the MF 0.14 μm trial

| Process parameters | Value |
|---|---|
| Feed flow rate | 9.5 m³/hour |
| Transmembrane pressure | 1.35 bar |
| Temperature | 20-29° C. |
| Flow speed | 5.6 m/s |
| VCR (volumetric concentration ratio) | ca. 3.5 |

The diagram of the attached FIG. 2 shows the production curve in terms of litres produced over time and with respect to the filtering surface of the MF 0.14 μm trial.

Once the VCR (volumetric concentration ratio) of about 3.5 is reached, the 28 litres of MF 0.14 μm concentrate were added with 23 litres of osmotised water (MF/DF), continuing the filtering in order to increase the extraction of the molecules of interest in the permeate. The MF/DF process with a 0.14 micron membrane yielded 26.5 litres of permeate. The final volume of concentrate of 0.14 micron MF/DF is of about 25 litres. The mean flow rate of the permeate in DF increases to values of about 170 (L/m²h).

Ultrafiltration of the MF Permeate, with 6 kD Molecular Cut-Off

This operation uses, as feeder, a solution of 97 L, +hold-up volume (ca. 7 litres), for a total of about 104 L.

The UF operation aims to eliminate compounds of high molecular weight (proteins, colloidal compounds, pectins, waxes, fragments of cell walls, etc.).

A spiral-wound polyethersulfone polymer membrane (Osmonics, USA) is used, with a molecular size of 6 kD, 28 mil spacer and filtering surface of 8.36 m².

The process parameters of the UF trial are reported in Table 2 below.

TABLE 2
Process parameters of the UF 6 kD trial

| Process parameters | Value |
|---|---|
| Feed flow rate | 4.4 m³/hour |
| Transmembrane pressure | 4.7 bar |
| Temperature | 22-24° C. |
| Flow rate | 0.25 m/s |
| VCR (volumetric concentration ratio) | ca. 4.1 |

Once the VCR of about 4.1 is reached, to the 24 litres of concentrate (+hold-up volume) is added 21 litres of osmotised water four times (making a total of 84 litres added), continuing the filtering (UF/DF) to increase the yield. A total of 95 litres of permeate of UF/DF 6 kD are obtained and 13 litres +hold-up volume of the UF/DF 6 kD concentrate.

The diagram of FIG. 3 attached shows the production curve in terms of litres produced over time and with respect to the filtering surface of the UF and UF/DF 6 kD trials.

### Reverse Osmosis (RO) Operation on the UF Permeate

The UF permeates and UF/DF ones are treated in RO in order to concentrate the product rich in antioxidant substances, and to eliminate the water, as permeate.

80 litres of UF 6 kD permeate along with 95 litres of UF/DF 6 kD permeate (+hold-up volume of about 10 litres), for a total of 185 L, were concentrated in RO with a pilot apparatus equipped with a polymer module made of composite spiral-wound high saline rejection polyamide, of the DE SAL company (USA), with a filtering surface of 7.0 m². The process parameters are reported in Table 3 below.

TABLE 3
Process parameters of the RO trial

| Process parameters | Value |
|---|---|
| Feed flow rate | 0.65-0.60 m³/hour |
| Operating pressure | 20-26 bar |
| Temperature | 22-27° C. |
| Mean permeate flow | 22 (L/m²h) |
| Salinity expressed in mg/l | 0-56 |
| VCR (volumetric concentration ratio) | 15.0 |

Membrane productivity remains constant in the region of 18.8 L/m² per hour throughout the trial.

During the trial, the pressure—initially set at 20 bar—automatically rises in the apparatus up to 26 bar after 40 minutes, owing to an increase in permeate salinity. For this reason, productivity at 26 bar increases to 23.14 L/m² per hour.

The RO trial lasted for a total of 65 minutes, in which no loss of permeability of the module was observed, which means that this membrane can operate without washing for long periods (months).

The liquid concentrated product is bright green due to the presence of chlorophyll.

As shown by the process diagram of FIG. 1, the RO permeate is reused in order to prepare the new extraction bath, that is, to prepare the decoction, avoiding the use of tap or well water.

### Drying of the RO Concentrate

The liquid product obtained as a retentate of the preceding phase is then turned into powder by a spray-drying process by means of a type ICF Lab 25 laboratory spray-dryer with an evaporation capacity of 500 ml/h.

The working conditions of the spray-dryer are the following:

pump flow rate: 5 ml/minute;

spray-drying chamber temperature: 90° C.;

exiting air temperature: 85° C.;

vacuum: 30 mbar.

The RO concentrate undergoing spray-drying yielded a yellowish green solid powdery product containing about 5% humidity. This powder may be further dried in an oven, and must be protected from humidity, given its hygroscopic nature.

The possibility of obtaining solid extracts of various concentrations was also studied, with the addition of dextrans or maltodextrins, or inert powders such as silica, according to the purpose.

### Membrane Washing Cycle

At the end of the work cycle, the membrane modules must be reconditioned in order to carry out the next work cycle. This allows removing any deposits forming on the membrane in order to restore the best permeate flows, that is, the productivity of these filters. To wash MF ceramic membranes it is necessary to first rinse the module with tap water for 5 minutes and then chemical wash it with a base solution (0.5 M soda) at 35° C. for 25 minutes in continuous mode. A second wash is then carried out with water to eliminate the chemical reactive until neutralisation of the washing solution is obtained.

For UF, the rinsing operation is to be carried out with tap water for 5-10 minutes, without recycling the water, followed by treatment with 0.25 M soda for 15 minutes. At the end of this operation, the module is washed with tap water and its permeability is checked.

For RO, the washing protocol simply envisages rinsing with tap water, followed by washing with distilled water.

### Chromatographic Analysis of the Dried RO Concentrate

The chromatogram of the powder obtained by drying the RO concentrate is shown in FIG. 4 attached.

Each molecule is quantified individually and the titre of the extract is defined as total caffeoylquinic derivatives and as total flavonoids, besides the total polyphenols present.

Table 4 below shows the quantitative results of the previously identified individual molecules relative to the starting aqueous extract and to the RO concentrate after the membrane-based separation process. The data are obtained by HPLC/DAD analysis.

TABLE 4
Individual compounds in artichoke extracts, ppm

| | Aqueous | RO |
|---|---|---|
| | extract | Conc. |
| Chlorogenic acid | 262.7 | 466.7 |
| MCQs (mono-caffeoylquinics) | 496.0 | 895.4 |
| Cynarin | 46.0 | 81.3 |
| Other DCQs (di-caffeoylquinics) | 42.0 | 38.0 |
| luteolin 7-O-rutinoside | 47.9 | 63.9 |
| luteolin 7-O-glucoside | 14.0 | 21.0 |
| luteolin 7-O-malonyl glucoside | 8.5 | 13.0 |
| Luteolin | 2.9 | 3.3 |
| TOTAL POLYPHENOLS | 920.7 | 1582.6 |

The anti-free radical capacity of the extract that can be correlated to the antioxidant properties is evaluated spectrophotometrically by means of the stable radical DPPH. Previous studies on artichoke leaves or bracts showed a dose-dependent effect, also correlated to the type of molecules present, on human LDL (Coinu et at 2006, loc. cit.).

Most of the cynarin present in the aqueous extracts of artichoke derives from 1,3-dicaffeoylquinic acid, initially found in the fresh plant following intramolecular trans-esterification caused by heating (Panizzi L. & Scarpati M. L., Gazz. Chim. Ital., 95, 71-82, 1965). 1,3-dicaffeoylquinic acid is the largest component of dicaffeoylquinic esters in hydroalcoholic extracts obtained cold, where the largest compound is the mono-substituted 5-caffeoylquinic derivative (chlorogenic acid).

Table 5 below reports the quantitative results of the single compounds analysed by HPLC/DAD obtained by spray-drying from the RO concentrate, considering Sample 1 as the powder as it is, and Sample 2 after adding 25 g/L of dextran to the RO concentrate.

TABLE 5
Individual powder compounds of dried RO concentrate, mg/g

| | Sample 1 | Sample 2 |
|---|---|---|
| Chlorogenic acid | 34.4 | 17.2 |
| MCQs | 64.8 | 32.6 |
| Cynarin | 5.5 | 2.9 |
| Other DCQs | 3.1 | 1.0 |
| Luteolin 7-O-rutinoside | 5.0 | 2.5 |
| Luteolin 7-O-glucoside | 2.0 | 1.0 |
| Luteolin 7-O-malonyl glucoside | 1.2 | 0.5 |
| Luteolin | 0.3 | 0.2 |
| TOTAL POLYPHENOLS | 116.3 | 57.9 |

Both the starting aqueous extract and all the intermediate solutions, including the RO concentrate and spray-drying product, were HPLC/DAD/MS analysed in order to establish the content in the caffeoylquinic derivatives and flavonoids present. The analytical and quantification conditions were standardised.

The previous results show how this type of process can yield both liquid and powder extracts of different contents in polyphenol compounds. The extracts can, in fact, come from a mixture of all the waste material of artichoke processing or it is possible to hypothesise the devising of a compressed air system that separates the stems and heads from the lighter tissues, consisting of bracts and leaves. In this way, processing the heads and stems will yield an extract rich in caffeoylquinic derivatives, while an extract rich in these compounds and in flavonoids can be obtained from the bracts and leaves.

The same technologies and extraction conditions can be applied to both cultivated and wild cardoon tissues in order to obtain extracts that are quantitatively and qualitatively different from those of the artichoke.

Specifically, the attached FIG. 5 shows a HPLC chromatogram of a hydroalcoholic extract of wild cardoon, which yields the specific proportions of the various polyphenol compounds present.

It is also worth specifying that the titre in the bioactive components of the previous extracts can be expressed both as the total content obtained spectrophotometrically and as the content in the three different subclasses (mono- and di-caffeoylquinic esters and flavonoids) evaluated by means of HPLC/DAD. As described above, each fraction may also be evaluated for the specific anti-free radical activity by means of DPPH that can be correlated to the antioxidant properties of the extract itself.

As may be noted from the above, one of the main advantages of the present invention is the possibility of valorising and reusing the large quantities of vegetal biomass resulting as waste from artichoke production, thereby obtaining products of high added value for the food and pharmaceutical industry, by means Of a process that respects the environment and ecosystem. negative anaerobic/aerobic bacteria.

The present invention has been disclosed with particular reference to some specific embodiments thereof, but it should be understood that modifications and changes may be made by the persons skilled in the art without departing from the scope of the invention as defined in the appended claims.

1. A process for the production of refined extracts from artichoke waste and from other plants of the Cynara genus, comprising, in sequence, the following operations:
a) obtaining a decoction of vegetable material from plants of the Cynara genus, in water, by means of hot infusion;
b) mechanically separating the vegetal material obtained in the previous phase from a filtered liquid phase;
c) treating the said filtered liquid phase resulting from the previous by means of tangential microfiltration (MF), to yield a retentate phase and a permeate phase;
d) treating the permeate coming from the previous operation by means of tangential ultrafiltration (UF), to yield a retentate phase and a permeate phase;
e) treating the permeate coming from operation d) by means of reverse osmosis (RO), to yield a retentate phase rich in purified polyphenol active ingredients and a permeate phase consisting of demineralised water;
the said vegetal material decoction obtained from the mechanical separation, being depleted of bitter polyphenol compounds, and being reusable as animal feed, and the said MF and UF retentate phases coming from operations c) and d) being reusable in the human food sector.

2. A process according to claim 1, wherein the said reverse osmosis retentate phase rich in purified polyphenol active ingredients obtained from operation e) undergoes spray-drying or freeze-drying.

3. A process according to claim 1, wherein the said vegetal material from which the decoction is obtained derives from the leaves, outer bracts, stems and heads of the artichoke, Cynara scolymus, either taken separately or intermixed.

4. A process according to claim 1, wherein the said vegetal material from which the decoction is obtained derives from the leaves, outer bracts, stems and heads of the cultivated cardoon, C. cardunculus L. var. altilis, either taken separately or intermixed.

5. A process according to claim 1, wherein the said vegetal material from which the decoction is obtained derives from the leaves, outer bracts, stems and heads of the wild cardoon, C. cardunculus L. var. sylvestris, either taken separately or intermixed.

6. A process according to claim 1, wherein the said hot infusion of phase a) is obtained by heating the said vegetal material in water at a temperature between 80° C. and 95° C., with a weight/volume ratio between 10 and 35%, for an overall time period of between 15 and 45 minutes.

7. A process according to claim 1, wherein the said mechanical separation operation b) of the vegetal material obtained from the previous phase is carried out by mechanical filtration on a 2-10 mm wire mesh filter.

8. A process according to claim 1, wherein after the said phase b) of mechanical separation, the separated vegetal material undergoes a second hot infusion phase followed by a further mechanical separation of the vegetal material obtained, in order to yield a filtered liquid phase.

9. A process according to claim 1, wherein the said operation c) of tangential microfiltration is carried out by means of spiral-wound polymer membranes or ceramic membranes with a molecular cut-off in the range 0.10-3.0 μm.

10. A process according to claim 9, wherein the said polymer membranes are made of polyethersulfone, regenerated cellulose acetate or nylon.

11. A process according to claim 9, wherein the said ceramic membranes for microfiltration are made of a tubular ceramic monolith in alumina with an internal coating of zirconia or titanium oxide.

12. A process according to claim 1, wherein downstream of the said operation c) of tangential microfiltration (MF) a diafiltration is carried out, feeding the membrane with demineralised water obtained from the said operation e) of reverse osmosis, that is added to the MF retentate.

13. A process according to claim 1, wherein the said operation d) of tangential ultrafiltration is carried out with polymer membranes with a molecular cut-off between 500 Dalton and 50 kDalton.

14. A process according to claim 13, wherein the said polymer membranes for UF are spiral-wound.

15. A process according to claim 1, wherein downstream of the said operation d) of tangential ultrafiltration (UF) a diafiltration is carried out, feeding the membrane with demineralised water obtained from the said operation e) of reverse osmosis, that is added to the UF retentate.

16. A process according to claim 1, wherein the said operation e) of reverse osmosis is carried out with spiral-wound polymer membranes with low or high saline rejection at an operating pressure between 7 and 50 bar.

17. A process according to claim 16, wherein the said spiral-wound polymer membranes have a mesh spacer with thickness ranging between 20 and 38 mil (0.51-0.97 mm), and size of the filtering modules 4 inches in diameter×40 inches in length (10.16 cm×101.6 cm), 6 inches in diameter×40 inches in length (15.24 cm×101.6 cm), or 8 inches in diameter×40 inches in length (20.32 cm×101.6 cm).

18. A process according to claim 16, wherein the said polymer membranes are flat, spiral-wound, hollow-fibre or boxed membranes.

19. A process according to claim 2, wherein the said drying operation of the reverse osmosis (RO) retentate is carried out by spray-drying.

20. A process according to claim 19, wherein the said spray-drying operation is carried out after adding dextrans or maltodextrins to the said RO retentate.

21. Use of refined extracts based on purified polyphenol active ingredients obtainable from the process of claim 1 for the production of food, nutraceutic, pharmaceutical, phytotherapeutic and cosmetic products.

22. Use according to claim 21 of the refined extracts in liquid form as stabilisers for the food product.

23. Use according to claim 21 of the refined extracts in solid form as semi-finished components with antioxidant and anti-free radical activity for the cosmetics and dermatological products.
